(19) United States
(12) Patent Application Publication
(10) Pub. No.: US 2026/0108252 A1
(43) Pub. Date: Apr. 23, 2026

(54) LEFT ATRIAL APPENDAGE CLOSURE DEVICES

(71) Applicant: BOSTON SCIENTIFIC SCIMED, INC., Maple Grove, MN (US)

(72) Inventors: Patrick Willoughby, Shoreview, MN (US); Scott Andrew McCullough, Plymouth, MN (US); Nicolas Ball-Jones, Minneapolis, MN (US)

(73) Assignee: BOSTON SCIENTIFIC SCIMED, INC., MAPLE GROVE, MN (US)

(21) Appl. No.: 19/364,139

(22) Filed: Oct. 21, 2025

Related U.S. Application Data
(60) Provisional application No. 63/710,258, filed on Oct. 22, 2024.

(51) Int. Cl. A61B 17/12; A61B 17/00

(52) U.S. Cl. CPC A61B 17/1219; A61B 17/12031; A61B 17/12122; A61B 2017/00871; A61B 2017/00951

(57) ABSTRACT

A left atrial appendage closure (LAAC) device may be adapted for occluding a patient's left atrial appendage (LAA). The LAAC device includes a shape memory polymer foam body that is adapted to expand from a crimped configuration for delivery to an expanded configuration after delivery. The shape memory polymer foam body. An adhesive layer is disposed relative to the shape memory polymer foam body.

### CROSS REFERENCE TO RELATED APPLICATIONS

This application claims the benefit of priority of U.S. Provisional Application No. 63/710,258 filed Oct. 22, 2024, the entire disclosure of which is hereby incorporated by reference.

### TECHNICAL FIELD

The disclosure relates generally to medical devices and more particularly to medical devices that incorporate a shape memory foam component.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted for occluding a patient's left atrial appendage (LAA). The LAAC device includes a shape memory polymer foam body that is adapted to expand from a compressed configuration to an expanded configuration and an adhesive layer that is disposed relative to the shape memory polymer foam body.

Alternatively or additionally, the adhesive layer may include an adhesive adapted to preferentially adhere to tissue.

Alternatively or additionally, the adhesive layer may include an adhesive adapted to provide an inflammatory response in tissue.

Alternatively or additionally, the adhesive layer may be disposed on the shape memory polymer foam body.

Alternatively or additionally, the adhesive layer may extend into pores within the shape memory polymer foam body.

Alternatively or additionally, the adhesive layer may include an adhesive precursor that becomes an adhesive after exposure to blood.

Alternatively or additionally, the adhesive layer may be applied to the shape memory polymer foam body while the shape memory polymer foam body is in the expanded configuration.

Alternatively or additionally, the adhesive layer may be applied to the shape memory polymer foam body while the shape memory polymer foam body is in the compressed configuration.

Alternatively or additionally, the adhesive layer may be formed by dip coating the shape memory polymer foam body.

Alternatively or additionally, the shape memory polymer foam body undergoes a priming process prior to forming the adhesive layer.

Another example may be found in a left atrial appendage closure (LAAC) device that is adapted for occluding a patient's left atrial appendage (LAA). The LAAC device includes a polymeric foam body that is adapted to expand from a compressed configuration to an expanded configuration. The polymeric foam body includes a plurality of pores that have a smaller size when the foam body is in the compressed configuration and a larger size when the foam body is in the expanded configuration. An adhesive layer extends over at least part of the polymeric foam body.

Alternatively or additionally, the adhesive layer may be applied to the foam body when the foam body is in the expanded configuration.

Alternatively or additionally, the adhesive layer may extend into at least some of the plurality of pores.

Alternatively or additionally, the adhesive layer may be applied to the foam body when the foam body is in the compressed configuration.

Alternatively or additionally, the adhesive layer may cover the polymeric foam body.

Alternatively or additionally, the polymeric foam body may undergo an oxidative process prior to applying the adhesive layer.

Alternatively or additionally, the polymeric foam body may undergo a plasma process prior to applying the adhesive layer.

Alternatively or additionally, the polymeric foam body may undergo an isocyanate exposure process prior to applying the adhesive layer.

Another example may be found in a left atrial appendage closure (LAAC) device that is adapted for occluding a patient's left atrial appendage (LAA). The LAAC device includes a shape memory polymer foam body that is adapted to expand from a compressed configuration to an expanded configuration. The shape memory polymer foam body includes a first end, a second end and a circumferential surface extending between the first end and the second end. An adhesive layer extends over the circumferential surface.

Alternatively or additionally, the first end and the second end may be at least substantially free of the adhesive layer.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a schematic view of an illustrative LAAC device being disposed and then expanded within an LAA;

FIG. 3 is a schematic view of an illustrative shape memory polymer foam forming part of an LAAC device, shown in an expanded configuration;

FIG. 3A is an expanded view of a portion of FIG. 3;

FIG. 4 is a schematic view of the illustrative shape memory polymer foam of FIG. 3, shown in a compressed configuration;

FIG. 4A is an expanded view of a portion of FIG. 4;

FIG. 5 is a schematic cross-sectional view of a portion of an LAAC device having an adhesive layer formed on a shape memory polymer foam body of the LAAC device while the shape memory polymer foam body of the LAAC device is in an expanded configuration;

FIG. 6A is a schematic cross-sectional view of a portion of an LAAC device having an adhesive layer formed on a shape memory polymer foam body of the LAAC device while the shape memory polymer foam body of the LAAC device is in a compressed configuration; and

FIG. 6B is a schematic cross-sectional view of a portion of the LAAC device shown in FIG. 6A, with the shape memory polymer foam body of the LAAC device subsequently shown in an expanded configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A left atrial appendage closure (LAAC) device may be adapted for occluding a patient's left atrial appendage (LAA). The LAAC device includes a shape memory polymer foam body that is adapted to expand from a compressed configuration to an expanded configuration. An adhesive layer is disposed relative to the shape memory polymer foam body.

In some cases, the adhesive layer may include an adhesive adapted to preferentially adhere to tissue. In some cases, the adhesive layer may include an adhesive adapted to provide an inflammatory response in tissue. In some cases, the adhesive layer may be disposed on the shape memory polymer foam body and/or may extend into at least some pores formed within the shape memory polymer foam body. In some cases, the adhesive layer may include an adhesive precursor that becomes an adhesive after exposure to blood. The adhesive layer may be applied to the shape memory polymer foam body while the shape memory polymer foam body is in the expanded configuration. The adhesive layer may be applied to the shape memory polymer foam body while the shape memory polymer foam body is in the compressed configuration. In some cases, the adhesive layer may be formed by dip coating the shape memory polymer foam body. In some cases, the shape memory polymer foam body may undergo a priming process prior to forming the adhesive layer.

A left atrial appendage closure (LAAC) device may be adapted for occluding a patient's left atrial appendage (LAA). The LAAC device includes a polymeric foam body that is adapted to expand from a compressed configuration to an expanded configuration. The polymeric foam body includes a plurality of pores. The plurality of pores have a smaller size when the polymeric foam body is in the compressed configuration and a larger size when the polymeric foam body is in the expanded configuration. An adhesive layer extends over at least part of the polymeric foam body.

In some cases, the adhesive layer may be applied to the polymeric foam body when the polymeric foam body is in the expanded configuration. The adhesive layer may extend into at least some of the plurality of pores. In some cases, the adhesive layer may be applied to the foam body when the foam body is in the compressed configuration. The adhesive layer may cover part of the polymeric foam body. In some cases, all or nearly all of the polymeric foam body may be covered with the adhesive layer. In some cases, only a distal region of the polymeric foam body could be coated with the adhesive layer. The distal region of the polymeric foam body could be expanded and secured into the LAA as an anchor, then the foam behind it could be allowed to expand. This may be useful where multiple smaller foam plugs would be deployed. Another option is that just the proximal region of the polymeric foam body could be coated. This would allow for the foam to be partially expanded into the LAA and manipulated to adjust positioning. Once the desired position is set, the foam plug would be fully deployed and adhesive activated, securing the device in place. An option of free ends and adhesive in the middle is useful as well. This device would stick in the middle and “hourglass” out at the ends to fit the appendage variations. In some cases, how much of the polymeric foam body is coated with adhesive may vary depending on how much anchoring needs to be supplied by the adhesive. If the device has an additional anchoring system, then it may be possible to use less adhesive. The porosity of the polymeric foam body may also influence the adhesive usage.

In some cases, the polymeric foam body may undergo an oxidative process prior to applying the adhesive layer. In some cases, the polymeric foam body may undergo a plasma process prior to applying the adhesive layer. In some cases, the polymeric foam body may undergo an isocyanate exposure process prior to applying the adhesive layer.

A left atrial appendage closure (LAAC) device may be adapted for occluding a patient's left atrial appendage (LAA). The LAAC device includes a shape memory polymer foam body that is adapted to expand from a compressed configuration to an expanded configuration. The shape memory polymer foam body includes a first end, a second end and a circumferential surface extending between the first end and the second end. An adhesive layer extends over the circumferential surface. In some cases, the first end and the second end may be at least substantially free of the adhesive layer, meaning that at least eighty (80) percent of the first end and at least eighty (80) percent of the second end are free of adhesive.

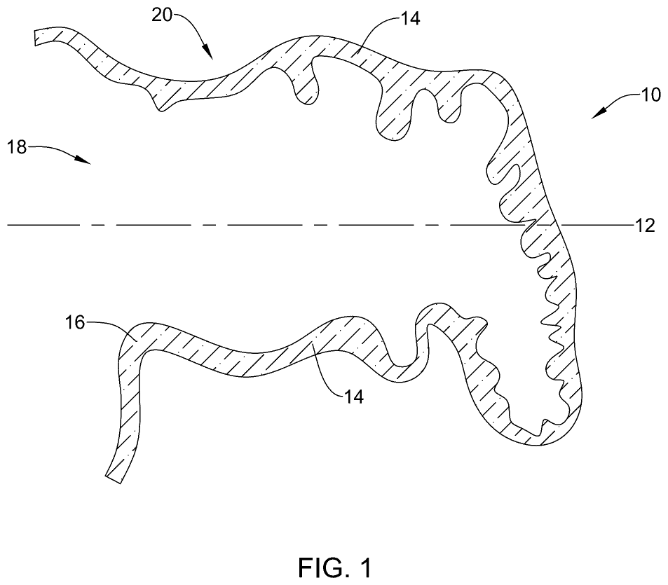

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

It will be appreciated that FIG. 1 shows an LAA 10 that is just one example of what a left atrial appendage may look like. While some patients may have a left atrial appendage that looks similar to the LAA 10, some patients have a left atrial appendage that may have a different shape from the LAA 10. The shape memory foam components described herein may be used in any possible left atrial appendage shape.

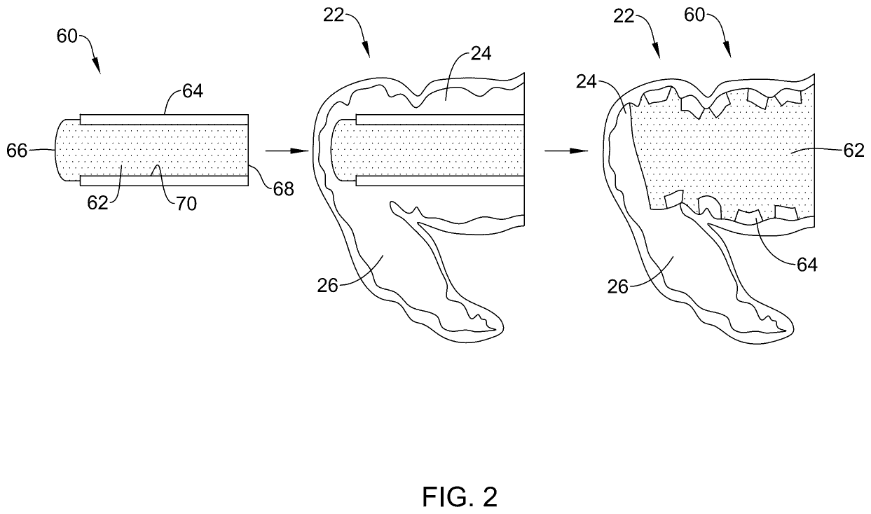

FIG. 2 shows an LAAC device 60 being implanted within an LAA 22 including a main segment 24 and a terminal segment 26. On the left side of FIG. 2, the LAAC device 60 is shown prior to implantation. The LAAC device 60 includes a shape memory polymer foam body 62 shown in a compressed configuration and an adhesive layer 64 that extends over at least part of the shape memory polymer foam body 62. The shape memory polymer foam body 62 includes a first end 66 and an opposing second end 68, with a circumferential surface 70 extending around the shape memory polymer foam body 62 from the first end 66 to the second end 68. In some cases, as shown, the adhesive layer 64 may only extend over the circumferential surface 70, and may not extend over the first end 66 or the second end 68. In some cases, not shown, the adhesive layer 64 may also extend over at least part of the first end 66 and/or the second end 68, depending on how the adhesive layer 64 is formed over or otherwise disposed on the shape memory polymer foam body 62.

In the middle of FIG. 2, the LAAC device 60 has been disposed within the main segment 24 of the LAA 22. At this point, the shape memory polymer foam body 62 of the LAAC device 60 is still in its compressed configuration. On the right side of FIG. 2, the shape memory polymer foam body 62 of the LAAC device 60 is now shown in its expanded configuration. As can be seen, the shape memory polymer foam body 62 of the LAAC device 60 spans across the main segment 24 of the LAA 22 when the shape memory polymer foam body 62 is in its expanded configuration. This pushes the adhesive layer 64 into contact with tissue on either side of the LAA 22. The adhesive layer 64 helps to secure the LAAC device 60 in position within the LAA 22 for a time until tissue ingrowth develops to further secure the LAAC device 60 in position.

There are options for how long the adhesive layer 64 remains effective in helping to secure the LAAC device 60 in position within the LAA 22. This may be influenced by the chemistry of the particular adhesive used within the adhesive layer 64. In some cases, if there is a discrete anchor present, then the chronic risk of the device embolizing is not a problem. In that case, the goal is to use the adhesive to affix the foam in the acute phase while clot and thrombus form to ensure that the foam does not contract as the adhesive dries, cures or solidifies. This may translate to a range of 4 to 12 hours as a reasonable window. In some cases, the adhesive may be selected to provide a mid-range duration between about 14 days to about 90 days. While the body responds to inflammatory processes quickly, that process is fully established at about 14 days and continues substantially out to about 90 days. A mid-range duration adhesive may be desired if there is a less rigorous primary anchoring mechanism. In situations in which the adhesive is the primary anchoring mechanism, an adhesive may be selected that will not substantially degrade for at least 90 days, and perhaps out to about 180 days to ensure full integration.

As will be appreciated, the shape memory polymer foam body 62 has a plurality of pores that are formed within the shape memory polymer foam body 62. The pores are relatively larger when the shape memory polymer foam body 62 is in its expanded configuration and are relatively smaller when the shape memory polymer foam body 62 is in its compressed configuration. Pore size may range from about 750 micrometers to about 2 millimeters when the shape memory polymer foam body 62 is expanded. In some cases, the pore size will vary within the shape memory polymer foam body 62. Pore size may be dependent upon the material construction of the shape memory polymer foam body 62 and how it is manufactured, among other factors. In some cases, it may be useful to refer to an average pore size when the shape memory polymer foam body 62 is in its expanded configuration versus an average pore size when the shape memory polymer foam body 62 is in its compressed configuration. As an example, the shape memory polymer foam body 62 may have an average pore size when expanded that is a magnitude of at least about 1.2 times greater, or at least about 1.3 times greater, or at least about 1.4 times greater, or at least about 1.5 times or more greater than when compressed.

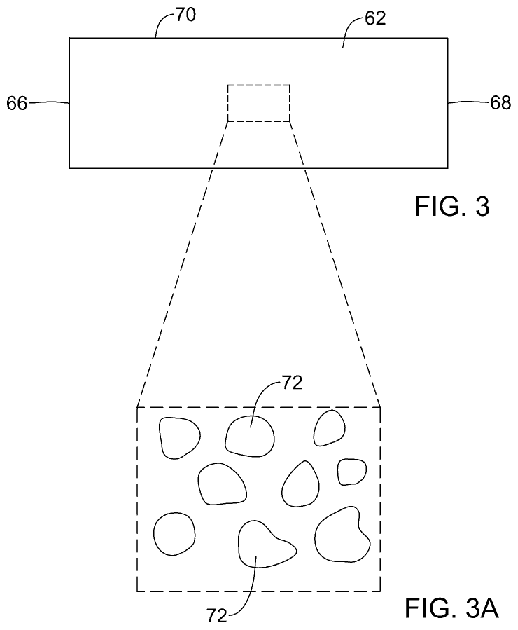

FIG. 3 is a schematic view of the shape memory polymer foam body 62 in its expanded configuration. As can be seen in FIG. 3A, which is an enlarged view of a small portion of the shape memory polymer foam body 62, the shape memory polymer foam body 62 includes a plurality of pores 72. In some cases, the adhesive layer 64 (shown in FIG. 2) may be applied while the shape memory polymer foam body 62 is in its compressed configuration or while the shape memory polymer foam body 62 is in its expanded configuration. This can impact how the adhesive layer 64 is dispersed over (and within) the shape memory polymer foam body 62.

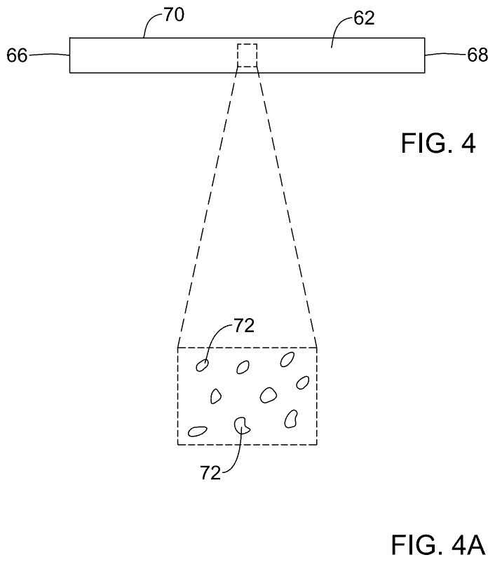

FIG. 4 is a schematic view of the shape memory polymer foam body 62 in its compressed configuration. As can be seen in FIG. 4A, which is an enlarged view of a small portion of the shape memory polymer foam body 62, the shape memory polymer foam body 62 includes a plurality of pores 72 that are squeezed closed due to the shape memory polymer foam body 62 being compressed. In some cases, the adhesive layer 64 (shown in FIG. 2) may be applied while the shape memory polymer foam body 62 is in its compressed configuration. This can impact how the adhesive layer 64 is dispersed over (and within) the shape memory polymer foam body 62. While the adhesive layer 64 extends over the shape memory polymer foam body 62, the adhesive layer 64 does not extend into the pores 72.

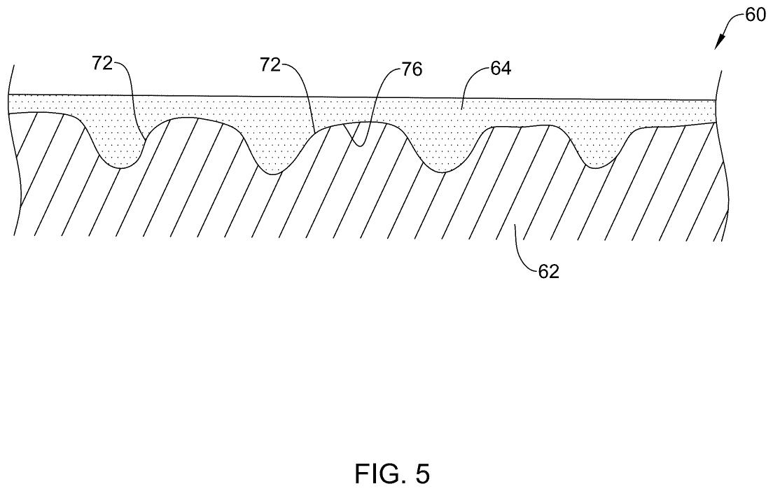

FIG. 5 shows a portion of the LAAC device 60, including the shape memory polymer foam body 62 and the adhesive layer 64. In this example, which shows the LAAC device 60 including the shape memory polymer foam body 62 in its expanded configuration, the adhesive layer 64 was applied while the shape memory polymer foam body 62 was in its expanded configuration. As a result, the adhesive layer 64 extends into the pores 72. In some cases, the adhesive layer 64 may extend into the pores 72 along an outer surface 76 of the shape memory polymer foam body 62, but may not extend substantially into pores 72 that are more interior within the shape memory polymer foam body 62. It will be appreciated that the outer surface 76 may include the circumferentially extending surface 70, and may optionally include at least part of the first end 66 and/or the second end 68.

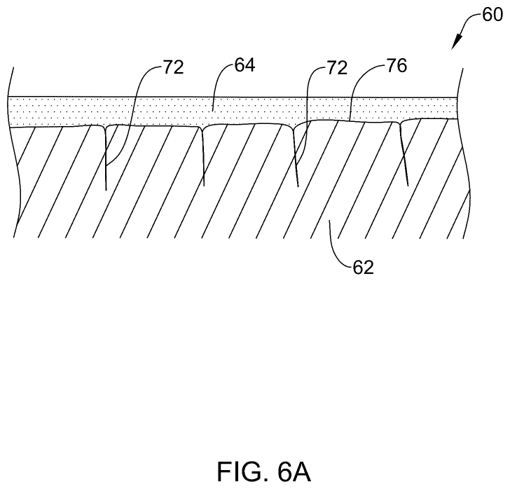

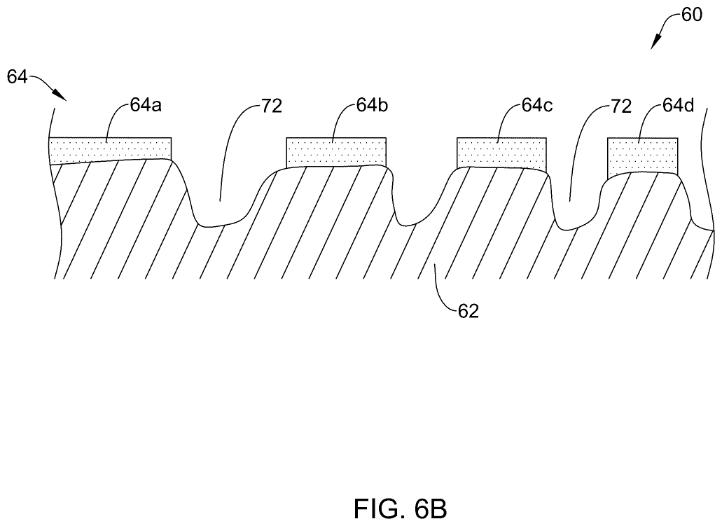

FIGS. 6A and 6B together provide an example of the LAAC device 60 in which the adhesive layer 64 was applied while the shape memory polymer foam body 62 was in its compressed configuration. As shown in FIG. 6A, which shows the LAAC device 60 while the shape memory polymer foam body 62 is in its compressed configuration, the pores 72 are largely closed off while the adhesive layer 64 is formed on the outer surface 76. As a result, the adhesive layer 64 is not present within the pores 72, as can be seen in FIG. 6B. FIG. 6B shows the LAAC device 60 with the shape memory polymer foam body 62 in its expanded configuration. While the two-dimensional representation shows the adhesive layer 64 forming individual sections 64a, 64b, 64c and 64d, it will be appreciated that in the three dimensional LAAC device 60, the individual sections 64a, 64b, 64c and 64d may combine with others of the individual sections 64a, 64b, 64c and 64d at locations circumferentially displayed from the particular two-dimensional slice through the LAAC device 60 that is shown in FIG. 6B.

As noted, the adhesive layer 64 may be applied either in the expanded configuration or the compressed configuration of the shape memory polymer foam body 62. The adhesive layer 64 may be applied by dipping the shape memory polymer foam body 62 into a solution of the adhesive that will form the adhesive layer 64. A solution of the adhesive that will form the adhesive layer 64 may be sprayed or painted onto the shape memory polymer foam body 62, for example. In some cases, the adhesive may be an integral part of the polymeric makeup of the shape memory polymer foam body 62. In some cases, the adhesive layer 64 may be designed to only stick to tissue, and to not stick to any component of a delivery system. In some cases, this may be achieved by selecting a particular adhesive that will only stick to tissue. In some cases, this may be achieved by using a material that changes over time. An example is a material that is non-adhesive as synthesized, but becomes adhesive as a result of a relatively fast degradation that results in a leaving group, with the remaining molecules (minus the leaving group) being adhesive in nature.

In some cases, the adhesive layer 64 may include an adhesive chemistry that presents a stronger inflammatory response than the shape memory polymer foam used to create the shape memory polymer foam body 62. When applied only to the circumferential surface 70, this inflammatory response can encourage more bridging of tissue from the anatomy into the shape memory polymer foam body 62. In some cases, this may help to drive faster and stronger integration of the LAAC device 60 while preserving the first end 66 and the second end 68 (one of which will be the distal-facing end and other of which will be the proximal-facing end) and its desirable biological reactions when exposed to blood. In some cases, the adhesive chemistry may be chemically degraded after implantation. In some cases, this may provide for adhesive action and potential inflammatory response to be acute in nature, thereby allowing for tissue bridging and integration to secure the LAAC device 60 chronically.

A variety of different materials may be used as, or included within, the adhesive layer 64. In some cases, the adhesive layer 64 may include fibrin. The adhesive layer 64 may include a cyano-acrylate material. In some cases, excess isocyanate groups on the surface of the adhesive layer 64 may react with amine groups within tissue. Slow adhesion formation may allow for time to adjust the LAAC device 60. This may provide slow degradation rates as well. The adhesive layer 64 may be functionalized with isothiocyanates for reaction with amino acids within tissue. The adhesive layer 64 may include PEG-NHS, which is a PEG (polyethylene glycol) derivative that includes an N-hydroxysuccinimide group. In some cases, the adhesive layer 64 may involve an NHS ester leaving group directly on the polyurethane. In some cases, the shape memory polymer foam body 62 may be functionalized with diazo moieties. This may allow the foam to bind to aspartic acid and glutamic acid residues present within the tissue, leading to adhesion through covalent modification. The adhesive layer 64 may include materials used in bioconjugation, such as thiols, disulfides and linkers that can bind to N-terminal cysteine residues.

In some cases, the shape memory polymer foam body 62 may be processed to make the foam more receptive to attachment of the adhesive layer 64. Parts or all of the shape memory polymer foam body 62 may be exposed to conditions that yield sites for chemical grafting. This may include modifying the surface alone, or throughout the body of the shape memory polymer foam body 62. An example includes exposure to a bath including oxidative solutions such as a hydroxide solution to yield available carboxylic acid groups on the foam. Another example includes a plasma treatment, which can yield available carboxylic acid groups, hydroxyl groups and other oxygen-containing functional groups. Another example includes exposure to a multifunctional isocyanate solution, which can endcap free hydroxyl groups within the foam with reactive isocyanate functional groups. In some cases, a coating may be applied selectively to specific surface, even when dipping the foam, through grafting methods that only impact the surface of the foam. An example includes UV grafting.

NHS-EDC grafting is another example of a process that may be used. EDC is short for 1-ethyl-3-(-3-dimethylaminopropyl) carbodiimide hydrochloride. As noted above, NHS is N-hydroxysuccinimide. NHS-EDC grafting can yield carboxylic acids that are able to couple to amine-groups within tissue. There are several methods for performing NHS-EDC grafting:

A first method involves treating a shape memory polyurethane foam to yield carboxylic acid groups. This includes an oxidative treatment in a peroxide solution, followed by rinsing. The foam is then submerged in an NHS/EDC solution and is allowed to react. A second method includes radical initiation of polymerization. This includes using a photo-initiator to generate reactive sites on the foam. Sidechain branches may be polymerized using acrylate-based chemistry such as acrylic acid, PEG methacrylate, and others. The polymerized acrylate chain may be end-capped using an NHS/EDC solution. A third method includes isocyanate grafting. This includes submerging the foam in a diisocyanate solution, which results in free hydroxyl groups in the foam becoming end-capped. Next, the foam is submerged in a solution of polyol-containing carboxylic acid groups, which results in attaching carboxylic acid functional groups to the surface. The foam is then submerged in an NHS/EDC solution and allowed to react with the carboxylic acid functional groups.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (about 98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D, L-lactide, poly(beta-hydroxybutyrate), polygluconate including poly-D-gluconate, poly-L-gluconate, poly-D, L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha, omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine-and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted for occluding a patient's left atrial appendage (LAA), the LAAC device comprising:
a shape memory polymer foam body adapted to expand from a compressed configuration to an expanded configuration; and
an adhesive layer disposed relative to the shape memory polymer foam body.

2. The LAAC device of claim 1, wherein the adhesive layer comprises an adhesive adapted to preferentially adhere to tissue.

3. The LAAC device of claim 1, wherein the adhesive layer comprises an adhesive adapted to provide an inflammatory response in tissue.

4. The LAAC device of claim 1, wherein the adhesive layer is disposed on the shape memory polymer foam body.

5. The LAAC device of claim 1, wherein the adhesive layer extends into pores within the shape memory polymer foam body.

6. The LAAC device of claim 1, wherein the adhesive layer comprises an adhesive precursor that becomes an adhesive after exposure to blood.

7. The LAAC device of claim 1, wherein the adhesive layer is applied to the shape memory polymer foam body while the shape memory polymer foam body is in the expanded configuration.

8. The LAAC device of claim 1, wherein the adhesive layer is applied to the shape memory polymer foam body while the shape memory polymer foam body is in the compressed configuration.

9. The LAAC device of claim 1, wherein the adhesive layer is formed by dip coating the shape memory polymer foam body.

10. The LAAC device of claim 1, wherein the shape memory polymer foam body undergoes a priming process prior to forming the adhesive layer.

11. A left atrial appendage closure (LAAC) device adapted for occluding a patient's left atrial appendage (LAA), the LAAC device comprising:
a polymeric foam body adapted to expand from a compressed configuration to an expanded configuration;
the polymeric foam body comprising a plurality of pores, the plurality of pores having a smaller size when the foam body is in the compressed configuration and a larger size when the foam body is in the expanded configuration; and
an adhesive layer extending over at least part of the polymeric foam body.

12. The LAAC device of claim 11, wherein the adhesive layer is applied to the foam body when the foam body is in the expanded configuration.

13. The LAAC device of claim 12, wherein the adhesive layer extends into at least some of the plurality of pores.

14. The LAAC device of claim 11, wherein the adhesive layer is applied to the foam body when the foam body is in the compressed configuration.

15. The LAAC device of claim 14, wherein the adhesive layer covers the polymeric foam body.

16. The LAAC device of claim 11, wherein the polymeric foam body undergoes an oxidative process prior to applying the adhesive layer.

17. The LAAC device of claim 11, wherein the polymeric foam body undergoes a plasma process prior to applying the adhesive layer.

18. The LAAC device of claim 11, wherein the polymeric foam body undergoes an isocyanate exposure process prior to applying the adhesive layer.

19. A left atrial appendage closure (LAAC) device adapted for occluding a patient's left atrial appendage (LAA), the LAAC device comprising:
a shape memory polymer foam body adapted to expand from a compressed configuration to an expanded configuration, the shape memory polymer foam body including a first end, a second end and a circumferential surface extending between the first end and the second end; and
an adhesive layer extending over the circumferential surface.

20. The LAAC device of claim 19, wherein the first end and the second end are at least substantially free of the adhesive layer.
